(19) United States
(12) Patent Application Publication
(10) Pub. No.: US 2005/0089571 A1
(43) Pub. Date: Apr. 28, 2005

(54) Pharmaceutical formulation for the active ingredient budesonide

(75) Inventors: Thomas Beckert, Warthausen (DE); Jennifer Dressman, Frankfurt (DE); Markus Rudolph, Neu-Isenburg (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: ROEHM GMBH & CO.KG, Darmstadt (DE)

(21) Appl. No.: 10/501,236

(22) PCT Filed: Mar. 6, 2003
(86) PCT No.: PCT/EP03/02269

(30) Foreign Application Priority Data
Mar. 27, 2002 (DE) 102 14 002.2

(51) Int. Cl. A61K 9/24

(52) U.S. Cl. 424/471

(57) ABSTRACT

The invention relates to a pharmaceutical formulation containing essentially a) an inner layer which can optionally be applied to a core, with the active substance budesonide, bound with a binding agent; b) a middle layer with a polymer covering agent which is soluble in intestinal juice or retardant; and c) an outer envelope or outer layer which is resistant to stomach juice, said layers being able to contain in a manner known per se other pharmaceutically usual adjutants. The inventive formulation is characterised in that the binding agent is a polymer or a copolymer with acid groups and the formulation of the inner layer without the middle and outer layer releases the bound active ingredient in a release test according to USP XXIII monography <711> dissolution with apparatus 2 (addle) at a rotational speed of 100/min in a phosphate buffer pH 7.5 after 30 min to a value of more than 80%.

### FIELD OF THE INVENTION

The invention relates to a pharmaceutical formulation for the active ingredient budesonide

### PRIOR ART

Löfberg, R. describes in “Research and Clinical Forums, Vol. 15 (5), pages 92-96 (1993), budesonide formulations for oral therapy of “inflammatory bowel desase (IBD)”. Described therein are budesonide pellets consisting of a sugar core with a thin budesonide layer in an undefined, water-insoluble rate limiting polymer and a coating of Eudragite® L 100-55. The pellets can be packed into gelatin capsules which represent the actual pharmaceutical form.

WO 95/08323 describes budesonide pellets with controlled release profile and a process for producing them. To improve the solubility of budesonide, the active ingredient is applied to the pellet cores in a mixture of excipients. For this purpose, the active ingredient is suspended in an alcohol:water mixture of 0:100 to 20:80, and at least two parts of a suitable water-soluble excipient, e.g. α-lactose monohydrate, sucrose or monosodium citrate, are added to one part of the mixture. In order to obtain a suitable release profile, the budesonide cores are coated with a two-layer coating of, for example, Eudragite® L, S, RS and/or RL inside and Eudragit® RS/RL outside.

WO 97/00512 and U.S. Pat. No. 5,849,327 described pharmaceutical forms for release of active ingredients such as, for example, budesonide in the lower gastrointestinal tract. The pharmaceutical form comprises the active ingredient bound in crosslinked polymer particles which are additionally coated with Eudragit® 100 S (copolymer of methyl methacrylate and methacrylic acid) a microbially degradable polysaccharide. The particles are packed into capsules which may, for example, in turn be coated with Eudragit® 100 S.

WO 01/68058 relates to the use of a multilayer pharmaceutical form which is essentially composed of a) a core with an active pharmaceutical ingredient which may be, for example, budesonide, b) an inner coating of a copolymer or a mixture of copolymers which are composed of 85 to 98% by weight free-radical polymerized C₁- to C₄-alkyl esters of acrylic or methacrylic acid and 15 to 2% by weight (meth)acrylate monomers with a quaternary ammonium group in the alkyl radical, and c) an outer coating of a copolymer which is composed of 75 to 95% by weight free-radical polymerized C₁- to C₄-alkyl esters of acrylic or methacrylic acid and 5 to 25% by weight (meth)acrylate monomers with an anionic group in the alkyl radical, for producing a pharmaceutical form which in the USP release test for two hours at pH 1.2 and subsequent rebuffering to pH 7.0 releases the contained active ingredient to the extent of less than 5% in the period up to 2.0 hours after the start of the test and to the extent of 30 to 80% at the time eight hours after the start of the test. The outer coating may be of the Eudragit® FS type.

Object and Achievement

One problem with pharmaceutical formulations containing the active ingredient budesonide is the low solubility of the active ingredient. One way of improving the solubility is, according to WO 95/08323, to formulate budesonide using water-soluble excipients.

For this purpose it is necessary to suspend budesonide in an alcohol:water mixture of 0:100 to 20:80. This is regarded as disadvantageous because, at present, because of environmental and occupational safety considerations, avoidance of the use of organic solvents is always attempted.

In addition, the formulation must take place with water-soluble excipients, e.g. α-lactose monohydrate, sucrose or monosodium citrate, which may lead to unwanted side effects. A known example is lactose intolerance in patients suffering from bowel diseases such as ulcerative colitis.

One object was regarded as being the provision of a budesonide formulation which avoids the prior art disadvantages. The production is intended to be possible entirely without the use of organic solvents. Excipients for increasing the solubility, like those mentioned in WO 95/08323, should be substantially avoided in order to reduce the risk of intolerance.

The object is achieved by a
pharmaceutical formulation substantially comprising
a) an inner layer, which may where appropriate be applied to a core, with the active ingredient budesonide, bound in a binder
b) an intermediate layer with a polymeric coating agent which is soluble in intestinal juice or extends release,
c) an outer envelope which is resistant to gastric juice or an outer layer with a coating agent which is resistant to gastric juice
where the layers may comprise in a manner known per se further pharmaceutically usual excipients,
characterized in that
the binder is a polymer or copolymer with acidic groups, and the formulation of the inner layer without intermediate and outer layer releases the bound active ingredient in the release test according to USP XXIII monograph <711> “Dissolution” with apparatus 2 (paddle) with 100 revolutions/min in phosphate buffer of pH 7.5 (according to monograph “Intestinal Fluid, Simulated, TS” without addition of pepsin) to the extent of more than 80% after 30 min.
   Mode of Operation of the Invention

The pharmaceutical formulation according to the invention substantially comprises
a) an inner layer, which may where appropriate be applied to a core, with the active ingredient budesonide, bound in a binder
b) an intermediate layer with a polymeric coating agent which is soluble in intestinal juice or extends release,
c) an outer envelope which is resistant to gastric juice or an outer layer with a coating agent which is resistant to gastric juice
where the layers may comprise in a manner known per se further pharmaceutically usual excipients,
characterized in that
the binder is a polymer or copolymer with acidic groups, and the formulation of the inner layer without intermediate and outer layer releases the bound active ingredient in the release test according to USP XXIII monograph <711> “Dissolution” with apparatus 2 (paddle) with 100 revolutions/min in phosphate buffer of pH 7.5 (according to monograph “Intestinal Fluid, Simulated, TS” without addition of pepsin) to the extent of more than 80% after 30 min.
   Inner Layer a)

The inner layer, which may where appropriate be applied to a core, comprises the active ingredient budesonide, bound in a polymeric binder with acidic groups.

The active ingredient budesonide is preferably employed in the commercially available micronized form. The average particle size may be, for example, in the range from 2 to 50 μm, preferably 5 to 25 μm, in particular 8 to 15 μm.

The active ingredient budesonide is bound in a polymeric binder with acidic groups. The binding of the active ingredient in the polymeric binder is intended preferably to take place without the use of organic solvents.

The polymeric binder with acidic groups may be, for example, a water-soluble polymer which can be applied in the form of a dispersion together with the active ingredient and, where appropriate, further excipients for example by spray application. It is possible in this way for example to provide pellets with an active ingredient-containing budesonide coating.

The polymeric binder with acidic groups may also be for example a polymer which can be thermally plasticated and which is melted in the presence of the active ingredient and, where appropriate, further excipients, or into a melt of which the active ingredient and, where appropriate, the further excipients are put. It is possible for example to produce active ingredient-containing sheets and to seal cores therein, or to apply the formulation of layer a) by spray application in the molten state.

Processing in this case can take place for example by injection molding or extrusion. The mixture can be converted into the form of granules for example by hot cut.

Polymeric Binder with Acidic Groups

Any pharmaceutically usable polymeric binder with acidic groups which, in combination with the bound active ingredient, leads to release of more than 80% of the bound budesonide after 30 min in the release test according to USP XXIII monograph <711> “Dissolution” with apparatus 2 (paddle) at 100 revolutions/min in phosphate buffer of pH 7.5 according to monograph “Intestinal Fluid, Simulated, TS” without addition of pepsin, is suitable for the purposes of the invention. This is possible only if there is an interaction between polymeric binders with acidic groups and the budesonide which increase the solubility of the budesonide. The exact molecular mechanism of the increase in solubility in this connection is unknown. It is merely assumed that the acidic groups are involved therein.

Those particularly suitable are
polymeric binders which are (meth)acrylate copolymers which comprise 40 to 95% by weight free-radical polymerized units of C₁- to C₄-alkyl esters of acrylic or methacrylic acid and 5 to 60% by weight (meth)acrylate monomers with an anionic group in the alkyl radical. The proportions mentioned can ordinarily add up to 100% by weight. However, it is also possible in addition, without this leading to an impairment or alteration of the essential properties, for small amounts in the region of 0 to 10, for example 1 to 5, % by weight of further monomers capable of vinylic copolymerization, such as, for example, methyl methacrylate, butyl methacrylate, butyl acrylate or hydroxyethyl methacrylate, to be present.

C₁- to C₄-alkyl esters of acrylic or methacrylic acid are in particular methyl methacrylate, ethyl methacrylate, butyl methacrylate, methyl acrylate, ethyl acrylate and butyl acrylate.

A (meth)acrylate monomer with an anionic group in the alkyl radical may be for example acrylic acid, but preferably methacrylic acid. The carboxyl groups may be up to 30 mol %, preferably up to 5 to 15 mol %, partially neutralized.

Anionic (meth)acrylate copolymers composed of 40 to 60, % by weight methacrylic acid and 60 to 40% by weight methyl methacrylate or 60 to 40% by weight ethyl acrylate (Eudragite® L or Eudragit® L 100-55 types) are suitable.

Equally suitable are anionic (meth)acrylate copolymers composed of 20 to 40% by weight methacrylic acid and 80 to 60% by weight methyl methacrylate (Eudragit® S type).

Likewise suitable are anionic (meth)acrylate copolymers composed of 20 to 34% by weight methacrylic acid and/or acrylic acid, 20 to 69% by weight methyl acrylate and 0 to 40% by weight ethyl acrylate and, where appropriate, 0 to 10% by weight further monomers capable of vinylic copolymerization, with the proviso that the glass transition temperature of the copolymer according to ISO 11357-2, subsection 3.3.3, is not more than 60° C. (Eudragit® type with medium methacrylic acid content).

The copolymer is composed in particular of free-radical polymerized units of
20 to 34, preferably 25 to 33, particularly preferably 28 to 32, % by weight methacrylic acid or acrylic acid, with preference for methacrylic acid,
20 to 69, preferably 35 to 65, particularly preferably 35 to 55, % by weight methyl acrylate and, where appropriate,
0 to 40, preferably 5 to 35, particularly preferably 15 to 35, % by weight ethyl acrylate, with the proviso that the glass transition temperature of the copolymer (without added plasticizer) according to ISO 11357-2, subsection 3.3.3, is not more than 60, preferably 40 to 60, particularly preferably 45 to 55° C.

The (meth)acrylate copolymer preferably consists essentially to exclusively of the monomers methacrylic acid, methyl acrylate and ethyl acrylate in the quantitative proportions indicated above. The proportions mentioned ordinarily add up to 100% by weight. However, it is also possible in addition, without this leading to an impairment or alteration of the essential properties, for small amounts in the region of 0 to 10, for example 1 to 5, % by weight of further monomers capable of vinylic copolymerization, such as, for example, methyl methacrylate, butyl methacrylate, butyl acrylate or hydroxyethyl methacrylate, to be present.

Said copolymers can be obtained in a manner known per se by free-radical bulk, solution, bead or emulsion polymerization. They must be brought before the processing by suitable grinding, drying or spraying processes into the particle size range according to the invention.

This can take place by simple crushing of extruded and cold pellets or hot cut.

The (meth)acrylate copolymer is preferably in the form of a dispersion, e.g. with a water content of from 60 to 80% by weight. The carboxyl groups may be up to 30 mol %, preferably from 5 to 15 mol %, partially neutralized by a base, e.g. NaOH.

Production of the inner layer a) preferably takes place by aqueous spraying of a budesonide-containing (meth)acrylate copolymer dispersion onto cores, e.g. sucrose pellets, with binding of the budesonide after the evaporation or volatilization of the water. The product temperature during the spray application can be for example 20 to 40, preferably 25 to 35° C. A release agent, e.g. talc, and a plasticizer, e.g. triethyl citrate, are normally added to the budesonide-containing (meth)acrylate copolymer dispersion. The processing of the budesonide and, where appropriate, of the additive can preferably take place by stirring into water with initially vigorous mixing, e.g. by mixing for example with a high-speed mixer (homogenizer) for 5 to 15 minutes. The suspension obtained in this way can then be added to the (meth)acrylate copolymer dispersion. The mixture should expediently be stirred continuously, and preferably also during the spraying process.

Also suitable are
polymeric binders which are vinylpyrrolidone/vinyl acetate copolymers. The molar proportion of vinyl acetate in this case is preferably in a range from 10 to 60 mol %, particularly preferably 30 to 50 mol % (suitable commercial products are, for example, Kollidon® VA64, BASF, Ludwigshafen, Germany).

However, the vinylpyrrolidone/vinyl acetate copolymers must usually be processed in the form dissolved a solvent, e.g. ethanol, which is less preferred.

Production of the inner layer a) can in this case take place by spraying a budesonide-containing vinyl-pyrrolidone/vinyl acetate copolymer solution, e.g. in ethanol, onto cores, e.g. sucrose pellets, with binding of the budesonide after evaporation of the solvent. The spraying temperature can in this case be for example from 30 to 60° C. A release agent, e.g. talc, and a plasticizer, e.g. triethyl citrate, are normally added to budesonide-containing vinylpyrrolidone/vinyl acetate copolymer solution.

Intermediate Layer b)

The intermediate layer consists essentially of a polymeric coating agent which is soluble in intestinal juice or extends release.

Polymeric Coating Agents Which are Soluble in Intestinal Juice

Suitable examples are (meth)acrylate copolymers which comprise 40 to 100% by weight free-radical polymerized units of C₁- to C₄-alkyl esters of acrylic or methacrylic acid and no or up to 60% by weight (meth)acrylate monomers with an anionic group in the alkyl radical.

These may be identical to the (meth)acrylate copolymers mentioned above for the inner layer a). The (meth)acrylate copolymers are preferably different from the (meth)acrylate copolymer of the inner layer.

Also suitable in addition are, for example, (meth)acrylate copolymers consisting of 10 to 30% by weight methyl methacrylate, 50 to 70% by weight methyl acrylate and 5 to 15% by weight methacrylic acid (Eudragit® FS type).

Release-Extending Polymeric Coating Agents

Release-extending polymeric coating agents are preferably used for the intermediate layer.

Suitable examples are (meth)acrylate copolymers which comprise 85 to 98% by weight free-radical polymerized units of C1- to C4-alkyl esters of acrylic or methacrylic acid and 15 to 2% by weight (meth)acrylate monomers with a quaternary ammonium group in the alkyl radical.

Appropriate (meth)acrylate copolymers are disclosed for example in EP-A 181 515 or in DE patent 1 617 751. They are polymers which are soluble or swellable independently of the pH and which are suitable for pharmaceutical coatings. A possible production method to be mentioned is bulk polymerization in the presence of a free-radical initiator dissolved in the monomer mixture. The polymer can also be produced likewise by solution or precipitation polymerization. The polymer can be obtained in this way in the form of a fine powder, achievable in the case of bulk polymerization by grinding and in the case of solution and precipitation polymerization for example by spray drying.

The (meth)acrylate copolymer is composed of 85 to 98% by weight free-radical polymerized C1- to C4-alkyl esters of acrylic or methacrylic acid and 15 to 2% by weight (meth)acrylate monomers with a quaternary ammonium group in the alkyl radical.

Preferred C1- to C4-alkyl esters of acrylic or methacrylic acid are methyl acrylate, ethyl acrylate, butyl acrylate, butyl methacrylate and methyl methacrylate.

The particularly preferred (meth)acrylate monomer with quaternary ammonium groups is 2-trimethylammoniumethyl methacrylate chloride.

A corresponding copolymer may be composed for example of 50-70% by weight methyl methacrylate, 20-40% by weight ethyl acrylate and 7-2% by weight 2-trimethyl-ammoniumethyl methacrylate chloride.

A specifically suitable copolymer comprises 65% by weight methyl methacrylate, 30% by weight ethyl acrylate and 5% by weight 2-trimethylammoniumethyl methacrylate chloride be composed (Eudragit® RS).

A further suitable (meth)acrylate copolymer may be composed for example of 85 to less than 93% by weight C1- to C4-alkyl esters of acrylic or methacrylic acid and more than 7 to 15% by weight (meth)acrylate monomers with a quaternary ammonium group in the alkyl radical. Such (meth)acrylate monomers are commercially available and have been used for a long time for release-extending coatings.

A specifically suitable copolymer comprises, for example, 60% by weight methyl methacrylate, 30% by weight ethyl acrylate and 10% by weight 2-trimethyl-ammoniumethyl methacrylate chloride (Eudragite® RL).

Also suitable in addition are, for example, neutral (meth)acrylate copolymers composed of 20 to 40% by weight ethyl acrylate and 60 to 80% by weight methyl methacrylate (Eudragit® NE type).

Blends

The preferred embodiment of layer b) are polymer blends. In particular, the Eudragit® RS 30 D and Eudragit® NE 30 D polymer types with relatively low permeability cause, even in layers of low thickness, a therapeutically unwanted great delay in delivery of active ingredient. For this reason, either the Eudragit® RL polymer type with higher permeability or blends of Eudragit® RL and Eudragit® RS, e.g. in the ratio 9:1 to 1:9, are preferably used for layer b). The Eudragit® NE polymer type with pore-forming additions such as, for example, NaCl, sucrose, hydroxypropyl-methylcellulose (HPMC) is also particularly suitable.

Further Polymers

To control delivery of active ingredient it may be advantageous in the individual case to admix further polymers. The content of further polymers in the blend is, however, not more than 20% by weight, preferably not more than 10% by weight, in particular 0-5% by weight, based on the (meth)acrylate copolymer.

Examples of such further polymers are: polyvinyl-pyrrolidone, polyvinyl alcohols, anionic (meth)acrylate copolymers composed of methyl methacrylate and/or ethyl acrylate and methacrylic acid (Eudragit® L 100, Eudragit® S 100, Eudragit® L 100-55). Anionic (meth)acrylate copolymers composed of methyl methacrylate, methyl acrylate and methacrylic acid, carboxymethyl-cellulose salts, hydroxypropylcellulose (HPMC), neutral (meth)acrylate copolymers composed of methyl methacrylate and ethyl acrylate (dry matter from Eudragit® NE 30 D), copolymers of methyl methacrylate and butyl methacrylate (Plastoid® B) or (meth)acrylate copolymers with quaternary ammonium groups (Eudragite® RL and Eudragite® RS).

Layer b) usually comprises further pharmaceutically customary excipients

Outer Layer c)

The outer layer c) may be an outer envelope which is resistant to gastric juice or an outer layer with a coating agent which is resistant to gastric juice. It has the task of preventing premature release of budesonide in the stomach.

Outer Envelope Which is Resistant to Gastric Juice

The outer envelope which is resistant to gastric juice may be a capsule. The capsule preferably consists essentially of gelatin or of hydroxypropycellulose and be provided in particular with a coating which is resistant to gastric juice.

The coating which is resistant to gastric juice of the capsule may be a (meth)acrylate copolymer which comprises 40 to 100% by weight free-radical polymerized units of C₁- to C₄-alkyl esters of acrylic or meth-acrylic acid and from 5 to 60% by weight (meth)acrylate monomers with an anionic group in the alkyl radical. The (meth)acrylate copolymer for the coating of the capsule may be identical or different from the copolymers of the inner and/or the intermediate layer.

The capsules comprise the active ingredient in the form of pellets or granules contains. The pellets or granules accordingly consist of the inner active ingredient-containing layer a) and of the intermediate layer b) which is soluble in intestinal juice or extends release. After the capsule has dissolved in the upper sections of the intestine, the contained pellets or granules are released.

Outer Layer c) With a Coating Agent Which is Resistant to Gastric Juice

In place of a filled capsule, a formulation may also be in the form for example of pellets in tablet form.

The outer coating agent which is resistant to gastric juice may be a (meth)acrylate copolymer which comprises 40 to 100% by weight free-radical polymerized units of C₁- to C₄-alkyl esters of acrylic or methacrylic acid and from 5 to 60% by weight (meth)acrylate monomers with an anionic group in the alkyl radical.

The (meth)acrylate copolymer may be identical or different from the copolymers of the inner and/or of the intermediate layer. It is preferably different from the (meth)acrylate copolymer of the intermediate layer.

Layer c) also usually comprises further customary pharmaceutical excipients.

Core Materials:

Cores which are optionally employed according to the invention are active ingredient-free pellets or minitablets in the particle size range between 10 to 3000 μm, preferably 100 to 1000 μm. Pellets preferably consist of sucrose, lactose or cellulose and are produced by powder layering or by the wet extrusion process with subsequent spheronization and final drying. Sucrose pellets are preferably employed.

Production of Layers b) and c):

The production of layers b) and c) takes place by processes customary in pharmaceutical technology, preferably by spray application. However, it is also possible to apply layers b) and c) by melt processing as also for layer a). It is possible for example to produce active ingredient-containing sheets and to seal cores therein, or to apply the layer by spray application in the molten state.

### EMBODIMENT BASED ON WO 01/68058

An embodiment based on WO 01/68058 is preferred. It is possible in this way to provide a budesonide pharmaceutical form which delivers virtually no active ingredient in the stomach and makes it possible for the active ingredient to be delivered uniformly and long-term in the intestine, especially shortly before or only in the region of the large intestine. The mode of active ingredient delivery is intended in particular to satisfy the requirement that in the USP release test for two hours at pH 1.2 and subsequent rebuffering to pH 7.0 the release of the contained active ingredient in the period up to 2.0 hours after the start of the test is less than 5% and at the time eight hours after the start of the test is 30 to 80%.

A difference from WO 01/68058 is according to the invention that the inner layer a) is applied to the core which comprises the active ingredient budesonide bound in a polymeric binder with acidic groups. The increased budesonide solubility which is achieved in this way results in an even more advantageous embodiment.

Intermediate Layer b) According to WO 01/68058

An intermediate layer b) of a copolymer or a blend of copolymers which are composed of 85 to 98% by weight free-radical polymerized C1- to C4-alkyl esters of acrylic or methacrylic acid and 15 to 2% by weight (meth)acrylate monomers with a quaternary ammonium group in the alkyl radical follows according to WO 01/68058.

A suitable copolymer may be produced for example from 93 to 98% by weight free-radical polymerized C1- to C4-alkyl esters of acrylic or methacrylic acid and 7-2% by weight 2-trimethylammoniumethyl methacrylate chloride. It is moreover possible for example for 50-70% by weight methyl methacrylate, 20-40% by weight ethyl acrylate to be present.

A corresponding copolymer is composed for example of 65% by weight methyl methacrylate, 30% by weight ethyl acrylate and 5% by weight 2-trimethylammoniumethyl methacrylate chloride (Eudragit® RS).

A further suitable copolymer can be produced for example from 85 to less than 93% by weight free-radical polymerized C1- to C4-alkyl esters of acrylic or meth-acrylic acid and more than 7 to 15% by weight 2-trimethylammoniumethyl methacrylate chloride.

A suitable copolymer is composed of 60% by weight methyl methacrylate, 30% by weight ethyl acrylate and 10% by weight 2-trimethylammoniumethyl methacrylate chloride (Eudragit® RL).

The proportionate amount of layer b) should be in the range from 2 to 20% by weight based on the core with the active ingredient. It is beneficial to use simultaneously both of the above mentioned copolymer types, preferably those having 5 and having 10% by weight 2-trimethylammoniumethyl methacrylate chloride (Eudragit® RS and Eudragit® RL) in blend. The ratio in the blend can be for example from 20:1 to 1:20, preferably 10:1 to 1:10.

Outer Layer c) According to WO 01/68058

An outer layer c) of a copolymer which is composed of 75 to 95% by weight free-radical polymerized C₁— to C₄-alkyl esters of acrylic or methacrylic acid and 5 to 60% by weight (meth)acrylate monomers with an anionic group in the alkyl radical follows to produce a pharmaceutical form which in the USP release test for two hours at pH 1.2 and subsequent rebuffering to pH 7.0 releases the contained active ingredient in the period up to 2.0 hours after the start of the test to the extent of less than 5% and at the time eight hours after the start of the test to the extent of 30 to 80%. For the therapy of ulcerative colitis, the outer coating may preferably be of the Eudragit® FS type. For the therapy of Crohn's disease, which may also occur even in sections of the small intestine, the outer coating may preferably be of the Eudragit® L type. Likewise suitable are anionic (meth)acrylate copolymers composed of 20 to 34% by weight methacrylic acid and/or acrylic acid., 20 to 69% by weight methyl acrylate and 0 to 40% by weight ethyl acrylate and, where appropriate, 0 to 10% by weight further monomers capable of vinylic copolymerization, with the proviso that the glass transition temperature of the copolymer according to ISO 11357-2, subsection 3.3.3, is not more than 60° C. (Eudragit® type with medium methacrylic acid content).

The proportional amount of the outer coating c) should be in the range from 10 to 50% by weight based on the weight of the core with the active ingredient and the inner coating.

Excipients Customary in Pharmacy

Layers a), b) and c) may comprise further pharmaceutically customary excipients in a manner known per se.

To produce the pharmaceutical form it is possible to employ pharmaceutically customary excipients in a manner known per se. These excipients may be present in the core or in the coating agent.

Dryers (Non-Stick Agents):

Dryers have the following properties: they have large specific surface areas, are chemically inert, are free-flowing and comprise fine particles. Because of these properties, they reduce the tack of polymers containing polar comonomers as functional groups.

Examples of dryers are:
Alumina, magnesium oxide, kaolin, talc, silica (Aerosils), barium sulfate and cellulose.
   Release Agents

Examples of release agents are:
esters of fatty acids or fatty amides, aliphatic, long-chain carboxylic acids, fatty alcohols and esters thereof, montan waxes or paraffin waxes and metal soaps; particular mention should be made of glycerol monostearate, stearyl alcohol, glycerol behenic acid ester, cetyl alcohol, palmitic acid, canauba wax, beeswax etc. The usual proportionate amounts are in the range from 0.05% by weight to 5, preferably 0.1 to 3, % by weight based on the copolymer.
   Further Excipients Customary in Pharmacy:

Mention should be made here of, for example, stabilizers, colorants, antioxidants, wetting agents, pigments, gloss agents etc. They are used in particular as processing aids and are intended can be to ensure a reliable and reproducible production process and good long-term storage stability. Further excipients customary in pharmacy may be present in amounts of from 0.001% by weight to 30% by weight, preferably 0.1 to 10% by weight, based on the copolymer.

Plasticizers:

Substances suitable as plasticizers ordinarily have a molecular weight between 100 and 20 000 and contain one or more hydrophilic groups in the molecule, e.g. hydroxyl, ester or amino groups. Citrates, phthalates, sebacates, castor oil are suitable. Examples of suitable plasticizers are alkyl citrates, propylene glycol, glycerol esters, alkyl phthalates, alkyl sebacates, sucrose esters, sorbitan esters, diethyl sebacate, dibutyl sebacate and polyethylene glycols 4000 to 20 000. Preferred plasticizers are tributyl citrate, triethyl citrate, acetyl triethyl citrate, dibutyl sebacate and diethyl sebacate. The amounts used are between 1 and 60, preferably 2 to 20, % by weight based on the film-forming polymer.

Multiparticulate Pharmaceutical Form

A further preferred embodiment is the multiparticulate pharmaceutical form described below.

The multiparticulate pharmaceutical form brings about an advantageous, substantially uniform release of budesonide in the small intestine and in the large intestine and comprises at least two different types of pellets, one type of pellet releasing the active ingredient predominantly in the pH range of the small intestine and the other predominantly in the pH range of the large intestine.

A suitable multiparticulate pharmaceutical form may comprise for example two forms of pellets A and B. The inner layer a) with the bound budesonide is present on a core, with pellet types A and B having two different polymer coatings, intermediate layers b), which determine the release of the active ingredient at different pH values.

Pellet form A can be provided with a polymer coating which makes continuous release of active ingredient possible, and an outer coating which is resistant to gastric juice and which rapidly dissolves above approximately pH 5.5. The outer coating of pellet form A can be, for example, Eudragit® L 100-55.

Pellet form B can be provided with a polymer coating, intermediate layer b), which in the USP release test at pH 6.8 releases less than 20% of the active ingredient in 6 hours and at pH 7.2 releases more than 50% of the active ingredient in 6 hours.

The multiparticulate drug form may be in the form of a capsule filled with pellets, e.g. a gelatin capsule, or it may be a tablet in which the pellets have been compressed together with conventional excipients to give the tablet unit.

The multiparticulate drug form is suitable for substantially uniform release of an active pharmaceutical ingredient in the small intestine and in the large intestine and comprises at least two forms of pellets, A and B, which comprise an active pharmaceutical ingredient in the core, but have different polymer coatings which determine the release of the active ingredient at different pH values. In vitro, the USP release test (USP 23, method 2) results at pH 6.8 and at pH 7.2 in combined profiles which are between the individual release curves for the two pellet forms A and B. In vivo, the release profile of pellet form A predominates in the small intestine, and release of active ingredient from pellet form B starts while in the large intestinal region.

The pellet cores consist entirely or partly of an active pharmaceutical ingredient. The cores are usually spherical or round and have diameters in the range from about 0.3 to 2 mm. The polymer coatings are in the range from about 2 to 16 mg of polymer per cm² surface area of the cores.

Pellet Form A

Pellet form A is provided with an inner polymer coating and an outer polymer coating.

Inner Polymer Coating

The inner polymer coating enables substantially pH-independent continuous release of active ingredient. The aim is an active ingredient release profile with which, in the USP release test (USP 23, method 2), at pH 6.8 there is about 40 to 70%, preferably 40 to 60%, release of active ingredient after 2 hours, and 60 to 100%, preferably 80 to 100% release after 4 hours. This is derived from the average residence time in the small intestine, which is about 4 hours.

The inner polymer coating of pellet form A may consist of a (meth)acrylate copolymer, of free-radical polymerized C1- to C4-alkyl esters of acrylic or methacrylic acid and (meth)acrylate monomers with a quaternary ammonium group in the alkyl radical.

Appropriate (meth)acrylate copolymers are disclosed, for example, in EP-A 181 515 or DE patent 1 617 751. They are polymers which are soluble or swellable independently of the pH and which are suitable for pharmaceutical coatings. A possible production process to be mentioned is bulk polymerization in the presence of a free-radical initiator dissolved in the monomer mixture. The polymer can likewise also be produced by a solution or precipitation polymerization. The polymer can be obtained in this way in the form of a fine powder, which is achievable in the case of bulk polymerization by grinding, and in the case of solution and precipitation polymerization for example by spray drying.

The (meth)acrylate copolymer is composed of 85 to 98% by weight free-radical polymerized C1- to C4-alkyl esters of acrylic or methacrylic acid and 15 to 2% by weight (meth)acrylate monomers with a quaternary ammonium group in the alkyl radical.

Preferred C1- to C4-alkyl esters of acrylic or meth-acrylic acid are methyl acrylate, ethyl acrylate, butyl acrylate, butyl methacrylate and methyl methacrylate.

The particularly preferred (meth)acrylate monomer with quaternary ammonium groups is 2-trimethylammoniumethyl methacrylate chloride.

A further suitable (meth)acrylate copolymer may be composed, for example, of 85 to less than 93% by weight C1- to C4-alkyl esters of acrylic or methacrylic acid and more than 7 to 15% by weight (meth)acrylate monomers with a quaternary ammonium group in the alkyl radical. Such (meth)acrylate monomers are commercially available and have been used for a long time for release-extending coatings (type (Eudragit® RL).

A specifically suitable copolymer comprises, for example, 60% by weight methyl methacrylate, 30% by weight ethyl acrylate and 10% by weight 2-trimethyl-ammoniumethyl methacrylate chloride (Eudragit® RL).

The desired release characteristics can be achieved for example through the thickness of the coating layer of polymer coatings of the “Eudragit® RL type” described above. This is achieved for example with a 5 to 15% coating of Eudragit® RL on active ingredient-containing cores with a diameter of 0.8 to 1.2 mm. The required release characteristics can also be composed with other layer thicknesses by admixing a copolymer composed of 50-70% by weight methyl methacrylate, 20-40% by weight ethyl acrylate and 7-2% by weight 2-trimethyl-ammoniumethyl methacrylate chloride (“Eudragit® RS type”). A specifically suitable copolymer comprises 65% by weight methyl methacrylate, 30% by weight ethyl acrylate and 5% by weight 2-trimethylammoniumethyl methacrylate chloride be composed (Eudragit® RS) . The Eudragit® RL and RS types can be mixed for example in the ratios 10:1 to 1:10. Higher proportions of the “Eudragit® RL type” are preferred, e.g. 60 to 90% by weight in the mixture.

The inner polymer coating may also consist of a (meth)acrylate copolymer composed of 20 to 40% by weight ethyl acrylate and 60 to 80% by weight methyl meth-acrylate, ethylcellulose or polyvinyl acetate.

Outer Polymer Coating

The outer polymer coating is a coating which is resistant to gastric juice and which rapidly dissolves only above about pH 5.5. The coating is thus intended to prevent release of active ingredient in the substantially stomach, i.e. this is intended to be no more than 10, preferably only 5, % according to USP 23. On transit into the small intestine it is intended that the outer polymer coating dissolve rapidly so that the release characteristics from this time onwards are determined by the inner polymer coating. If the outer polymer coating is too thin, too much active ingredient is released in the stomach. If the outer polymer coating is applied too thickly, it prevents direct release of active ingredient in the small intestine. Suitable layer thicknesses are, for example, in the range from 15 to 150 μm, preferably, for example, at 20 to 60 μm. Based on the weight of the core provided with the inner polymer coating and having a diameter of from 0.8 to 1.25 mm, it is usually suitable to apply polymer (based on dry matter) in the range from 8 to 40% by weight, preferably from 10 to 25% by weight.

The polymer coating which is resistant to gastric juice of pellet form A may of a (meth)acrylate copolymer which contains acidic groups and has, for example, acrylic acid, but preferably methacrylic acid, residues.

The (meth)acrylate copolymer consists of 40 to 100, preferably 45 to 99, in particular 85 to 95, % by weight free-radical polymerized C₁— to C₄-alkyl esters of acrylic or methacrylic acid and may comprise 0 to 60, preferably 1 to 55, in particular 5 to 15, % by weight (meth)acrylate monomers with an anionic group in the alkyl radical. C₁— to C₄-alkyl esters of acrylic or methacrylic acid are, in particular, methyl methacrylate, ethyl meth-acrylate, butyl methacrylate, methyl acrylate, ethyl acrylate and butyl acrylate.

Suitable examples are also neutral (meth)acrylate copolymers of 20 to 40% by weight ethyl acrylate and 60 to 80% by weight methyl methacrylate (Eudragit® NE type) if they are used in a mixture with (meth)acrylate copolymers containing acidic groups.

Particularly suitable (meth)acrylate copolymers are composed of 40 to 60% by weight methacrylic acid and 60 to 40% by weight methyl methacrylate or 60 to 40% by weight ethyl acrylate (Eudragit® L or Eudragit® L100-55 types).

Also suitable in principle are anionic (meth)acrylate copolymers of 20 to 40% by weight methacrylic acid and 80 to 60% by weight methyl methacrylate (Eudragit® S type).

Also suitable are (meth)acrylate copolymers consisting of 10 to 30% by weight methyl methacrylate, 50 to 70% by weight methyl acrylate and 5 to 15% by weight methacrylic acid (Eudragit® FS type).

The polymer coating which is resistant to gastric juice of pellet form A may also consist of shellac, HPMCP (hydroxypropylmethylcellulose phthalate), CAP (cellulose acetate phthalate), HPMC-AS (hydroxypropylmethyl-cellulose acetate succinate) or polyvinyl acetates. phthalate.

However, care must be taken in every case that the coating is adjusted for example in relation to layer thickness and, where appropriate, mixing with other polymers in such a way that it dissolves rapidly after transit into the small intestine.

Pellet Form B

Pellet form B releases, at pH 6.8 in the USP release test (USP 23, method 2), not more than 10%, preferably not more than 5%, after 2 hours and not more than 20, preferably not more than 10, % of the active ingredient after 4 hours. At pH 7.2, about 40 to 60% of active ingredient are released after 3 hours, and about 80 to 100 are released after 60 hours.

The polymer coating for pellet form B may be a (meth)acyrlate copolymer which is composed of 60 to 95% by weight free-radical polymerized C₁— to C₄-alkyl esters of acrylic or methacrylic acid and 5 to 40% by weight (meth)acrylate monomers with an acidic group in the alkyl radical.

Particular suitable (meth)acrylate copolymers consist of 10 to 30% by weight methyl methacrylate, 50 to 70% by weight methyl acrylate and 5 to 15% by weight methacrylic acid (Eudragite® FS type).

Likewise suitable are (meth)acrylate copolymers of 20 to 40% by weight methacrylic acid and 80 to 60% by weight methyl methacrylate (Eudragito® S type).

Uses

The pharmaceutical formulation of the invention can be used for the therapy of ulcerative colitis, Crohn's disease and/or other, especially inflammatory, disorders of the gastrointestinal tract which can be treated with budesonide.

Budesonide Content Per Dose Unit

The budesonide content, preferably micronized budesonide, per dose unit (pellet) may be for example from 0.5 to 30 mg, preferably 1 to 10 mg. A dose unit, a pellet-containing capsule or a tablet compressed from pellets, may comprise for example from 100 to 1000, preferably 150 to 750, pellets.

### EXAMPLES

The release test for budesonide is carried out in accordance with USP XXIII monograph <711> “Dissolution” with apparatus 2 (paddle) with 100 revolutions/min in phosphate buffer of pH 7.5 according to the monograph “Intestinal Fluid, Simulated, TS” without addition of pepsin or in purified water with the paddle at 100 revolutions/min with 500 ml of dissolving medium. 400 mg of sample were weighed for each determination. Detection took place by means of HPLC with a PP 18 column, 10 cm (Phenomenex) and UV detection at 246 nm. Equipment: pump L 7000 100 (from Merck-Hitachi, Darmstadt, Germany), autosampler L 7000 200 (from Merck-Hitachi, Darmstadt, Germany) UV/VIS detector L 4250 (from Merck-Hitachi, Darmstadt, Germany). The volume injected was 100 μl, and the flow rate was 1 ml/min. The retention times averaged 2.5 min. At the end of the test, the pellets were homogenized with an Ultra Turrax for 10 min. The content was used as 100% value in the calculation. 3 to 6 tests were carried out for each medium.

### Example 1

### Not According to the Invention: Determination of the Rate of Dissolution of Budesonide Without Binder

The active ingredient dissolves under the stated conditions in vitro in the following way:

Release in purified water and
phosphate buffer of pH 7.5
(% of theory)

| Time (min) | Mean | Rel. standard deviation |
|---|---|---|
| 0 | 0.0 | 0.0 |
| 60 | ca. 3.7 | 0.4 |
| 120 | ca. 5.4 | 1.0 |
| 180 | ca. 7.1 | 1.6 |

### Example 2

### According to the Invention: Embedding of Budesonide in a Binder on the Laboratory Scale

6 g of budesonide, 5 g of talc and 1 g of triethyl citrate are dispersed in 65 of purified water using a homogenizer (Ultra Turrax, from Jahnke & Kunkel, Germany) and, while stirring gently with a propeller stirrer, mixed with 33 g of Eudragits® L 30 D-55. This spray suspension is sprayed onto 500 g of sucrose pellets, 0.8×1.0 mm (from Werner, Tornesch, Germany) while agitating in a STREA 1 fluidized bed apparatus (from Aeromatic, Bubendorf, Switzerland).

The test is described by the following data:

| | |
|---|---|
| Coating dry matter (CDM) [g] | 10 |
| Plasticizer based on CDM | 10% |
| Release agent based on CDM | 50% |
| Solids content of dispersion (m/m) | 4.4% |
| CDM based on core mass | 2% |
| Coating apparatus | Strea 1 |
| Type of pellets | sucrose |
| Nozzle diameter [mm] | 0.8 |
| Spraying pressure [bar] | 0.5 |
| Batch size [g] | 500 |
| Amount applied [g] | 110 |
| Preheating time [min] | 5 |
| Spraying time [min] | 52 |
| Inlet air temperature [° C.] | 41 |
| Outlet air temperature [° C.] | 30 |
| Spraying rate [g/min] | 2.1 |
| After-drying time [min] | 10 |

The pellets release the active ingredient under the indicated conditions in vitro as described below. At the end of the test, the pellets were homogenized using an Ultra Turrax for 10 min, and the budesonide concentration in the solution was again determined. The latter measurement was used as 100% value (theoretically possible budesonide content in the solution) in the calculation.

| | |
|---|---|
| Release in phosphate | Release in purified |
| buffer of pH 7.5 | water |
| (% of theory) | (% of theory) |

| Time | | Rel. standard | | Rel. standard |
|---|---|---|---|---|
| (min) | Mean | deviation | Mean | deviation |
| 0 | 0.0 | 0.0 | 0.0 | 0.0 |
| 15 | 65.3 | 2.0 | 4.9 | 0.1 |
| 30 | 94.4 | 1.1 | 10.8 | 1.9 |
| 60 | 84.4 | 1.5 | 16.7 | 2.2 |
| 120 | 88.1 | 1.5 | 21.6 | 0.8 |

### Example 3

### According to the Invention: Embedding of Budesonide in a Binder on the Pilot-Plant Scale

36 g of budesonide, 60 g of talc and 12 g of triethyl citrate are dispersed in 632 of purified water using a homogenizer (Ultra Turrax, from Jahnke & Kunkel, Germany) and, while stirring gently with a propeller stirrer, mixed with 33 g of Eudragite® L 30 D-55. This spray suspension is sprayed onto 6000 g of sucrose pellets, 0.8×1.0 mm (from Werner, Tornesch, Germany) while agitating in a WSG 5 fluidized bed apparatus (from Glatt AG, Binzen, Germany).

The test is described by the following data:

| | |
|---|---|
| Coating dry matter (CDM) [g] | 120 |
| Plasticizer based on CDM | 10% |
| Release agent based on CDM | 50% |
| Solids content of dispersion (m/m) | 3.8% |
| CDM based on core mass | 2% |
| Coating apparatus | WSG 5 |
| Type of pellets | sucrose |
| Nozzle diameter [mm] | 2.0 |
| Spraying pressure [bar] | 2.0 |
| Batch size [g] | 6000 |
| Amount applied [g] | 1140 |
| Preheating time [min] | 5 |
| Spraying time [min] | 57 |
| Inlet air temperature [° C.] | 44 |
| Outlet air temperature [° C.] | 34 |
| Spraying rate [g/min] | 20.0 |
| After-drying time [min] | 10 |

The pellets release the active ingredient under the indicated conditions in vitro in the following way:

Release (% of theory)

| Time (min) | Mean | Standard deviation |
|---|---|---|
| 0 | 0.0 | 0.0 |
| 15 | 71.7 | 7.0 |
| 30 | 103.7 | 3.2 |
| 60 | 100.5 | 4.3 |
| 120 | 98.3 | 5.1 |

### Example 4

### Further Processing of Pellets from Example 2 by Application of a Release-Extending Layer b) and of a Layer C) Resistant to Gastric Juice Pharmaceutical Formulation or Pharmaceutical form Suitable for the Therapy of Ulcerative Colitis

Preparation of Spray Suspension 1 (Layer b)):

8.75 g of talc and 7 g of dibutyl sebacate are dispersed with a homogenizer (Ultra Turrax, from Jahnke & Kunkel, Germany) in 156.3 g of purified water and, while stirring gently with a propeller stirrer, mixed with a mixture of 26.3 g of Eudragit® RS 30 D and 8.8 g of Eudragit® RL 30 D.

350 g of pellets from example 2 were coated in a Strea 1 fluidized bed apparatus (from Aeromatic, Bubendorf, Switzerland) under the following conditions:

| | |
|---|---|
| Coating dry matter (CDM) [g] | 35 |
| Plasticizer based on CDM | 20% |
| Release agent based on CDM | 50% |
| Solids content of dispersion (m/m) | 17% |
| CDM based on core mass | 10% |
| Coating apparatus | Strea 1 |
| Type of pellets | example 2 |
| Nozzle diameter [mm] | 0.8 |
| Spraying pressure [bar] | 0.5 |
| Batch size [g] | 350 |
| Amount applied [g] | 297.5 |
| Preheating time [min] | 5 |
| Spraying time [min] | 117 |
| Inlet air temperature [° C.] | 40 |
| Outlet air temperature [° C.] | 329 |
| Spraying rate [g/min] | 2.5 |
| After-drying time [min] | 5 |

The coated cores then undergo after-drying on trays in a mechanical convection oven at 40° C. for 24 h.

Preparation of Spray Suspension 2 (Layer c)):

1.3 g of glycerol monostearate, 1.3 g of triethyl citrate and 0.5 g of polysorbate 80 are dispersed in 156.3 g of purified water using a homogenizer (Ultra Turrax, from Jahnke & Kunkel, Germany) and, while stirring gently with a propeller stirrer, mixed with 87.5 g of Eudragit® FS 30 D.

400 g of pellets from example 2 with a release-slowing coating of spray suspension 1 were coated in a Strea 1 fluidized bed apparatus (from Aeromatic, Bubendorf, Switzerland) under the following conditions:

| | |
|---|---|
| Coating dry matter (CDM) [g] | 30 |
| Plasticizer based on CDM | 3% |
| Release agent based on CDM | 5% |
| Solids content of dispersion (m/m) | 20% |
| CDM based on core mass | 7.5% |
| Coating apparatus | Strea 1 |
| Type of pellets | example 2 |
| Nozzle diameter [mm] | 0.8 |
| Spraying pressure [bar] | 0.5 |
| Batch size [g] | 400 |
| Amount applied [g] | 165 |
| Preheating time [min] | 5 |
| Spraying time [min] | 54 |
| Inlet air temperature [° C.] | 31 |
| Outlet air temperature [° C.] | 25 |
| Spraying rate [g/min] | 3.1 |
| After-drying time [min] | 5 |

The coated cores then undergo after-drying on trays in a mechanical convection oven at 40° C. for 2 h.

The pellets coated with layers b) and c) release the active ingredient under the indicated conditions in virto in the following way:

Release in phosphate buffer of pH 7.5
(% of theory)

| Time (min) | Mean | Rel. standard deviation |
|---|---|---|
| 0 | 0.0 | 0 |
| 30 | 0.0 | 0 |
| 60 | 0.0 | 0 |
| 120 | 1.8 | 0.1 |
| 180 | 4.2 | 0.3 |
| 240 | 11.7 | 1.8 |
| 300 | 32.6 | 2.0 |
| 360 | 50.5 | 7.4 |
| 480 | 73.3 | 7.0 |
| 600 | 85.3 | 3.0 |

1. A pharmaceutical formulation comprising
a) an inner layer, which may where appropriate be applied to a core, with the active ingredient budesonide, bound in a binder
b) an intermediate layer with a polymeric coating agent which is soluble in intestinal juice or extends release,
c) an outer envelope which is resistant to gastric juice or an outer layer with a coating agent which is resistant to gastric juice
where the layers may comprise in a manner known per se further pharmaceutically usual excipients,
wherein the binder is a polymer or copolymer with acidic groups, and the formulation of the inner layer without intermediate and outer layer releases the bound active ingredient in the release test according to USP XXIII monograph <711> “Dissolution” with apparatus 2 (paddle) with 100 revolutions/min in phosphate buffer of pH 7.5 to the extent of more than 80% after 30 min.

2. The pharmaceutical formulation as claimed in claim 1, wherein the polymeric binder is a (meth)acrylate copolymer which comprises 40 to 95% by weight free-radical polymerized units of C₁— to C₄-alkyl esters of acrylic or methacrylic acid and 5 to 60% by weight (meth)acrylate monomers with an anionic group in the alkyl radical.

3. The pharmaceutical formulation as claimed in claim 1, wherein the polymeric binder is a vinylpyrrolidone/vinyl acetate copolymer.

4. The pharmaceutical formulation as claimed in claim 1, wherein the intermediate layer is a (meth)acrylate copolymer which comprises 40 to 100% by weight free-radical polymerized units of C₁— to C₄-alkyl esters of acrylic or methacrylic acid and no or up to 60% by weight (meth)acrylate monomers with an anionic group in the alkyl radical.

5. The pharmaceutical formulation as claimed in claim 1, wherein the intermediate layer is a (meth)acrylate copolymer which comprises 85 to 98% by weight free-radical polymerized units of C1- to C4-alkyl esters of acrylic or methacrylic acid and 15 to 2% by weight (meth)acrylate monomers with a quaternary ammonium group in the alkyl radical.

6. The pharmaceutical formulation as claimed in claim 1, wherein the outer coating agent which is resistant to gastric juice is a (meth)acrylate copolymer which comprises 40 to 100% by weight free-radical polymerized units of C₁— to C₄-alkyl esters of acrylic or methacrylic acid and 5 up to 60% by weight (meth)acrylate monomers with an anionic group in the alkyl radical.

7. The pharmaceutical formulation as claimed in claim 1, wherein the outer envelope which is resistant to gastric juice is a capsule.

8. The pharmaceutical formulation as claimed in claim 6, wherein the capsule consists essentially of gelatin or of hydroxypropycellulose.

9. The pharmaceutical formulation as claimed in claim 6, wherein the capsule is provided with a coating which is resistant to gastric juice.

10. The pharmaceutical formulation as claimed in claim 6, wherein the pharmaceutical formulation comprises the active ingredient in the form of pellets or granules.

11. The pharmaceutical formulation as claimed in claim 1, wherein the pharmaceutical formulation is a multiparticulate pharmaceutical form with substantially uniform release of budesonide in the small intestine and in the large intestine, which comprises at least two different types of pellets, one type of pellet releasing the active ingredient predominantly in the pH range of the small intestine and the other predominantly in the pH range of the large intestine.

12. The pharmaceutical formulation as claimed in claim 11, wherein the pellets are enclosed in a capsule comprising (meth)acrylate copolymer which comprises 40 to 100% by weight free-radical polymerized units of C₁— to C₄-alkyl esters of acrylic or methacrylic acid and 5 up to 60% by weight (meth)acrylate monomers with an anionic group in the alkyl radical.

13. The pharmaceutical formulation as claimed in claim 11, wherein the pellets are in the form of a tablet in which the pellets have been compressed together with conventional excipients to give the tablet unit.

14. Process for producing a pharmaceutical formulation as claimed in claim 1, wherein
an inner layer a) in which budesonide is bound in a polymeric binder with acidic groups is produced in a manner known per se by spray application or melt processing, where the inner layer a) is where appropriate applied to a core, and subsequently the intermediate layer b) and the outer layer c) are applied in a manner known per se by spray application or melt processing.

15. The process for producing a pharmaceutical formulation as claimed in claim 14, wherein a binder comprising a (meth)acrylate copolymer which comprises 40 to 95% by weight free-radical polymerized units of C₁— to C₄-alkyl esters of acrylic or methacreylic acid and 5 to 60% by weight (meth)acrylate monomers with an anionic group in the alkyl radical is employed in the form of a dispersion, and the inner layer a) is produced by aqueous spraying of a budesonide-containing (meth)acrylate copolymer dispersion onto cores, with binding of the budesonide after evaporation of the water.

16. A method for treating ulcerative colitis, Crohn's disease and/or other disorders of the gastrointestinal tract which can be treated with budesonide, which comprises:
administering to a patient in need thereof an effective amount of the pharmaceutical formulation as claimed in claim 1.
